(19) United States
(12) Patent Application Publication
(10) Pub. No.: US 2004/0052852 A1
(43) Pub. Date: Mar. 18, 2004

(54) Carbohydrate-based delivery system for creatine and other bioactive ingredients

(76) Inventors: Michael Farber, Ville St. Laurent (CA); Jonathan Farber, Montreal (CA)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/416,815

(22) PCT Filed: Sep. 25, 2002
(86) PCT No.: PCT/CA02/01441

(30) Foreign Application Priority Data
Sep. 25, 2001 (CA) 2,358,045

(51) Int. Cl. A61K 9/14

(52) U.S. Cl. 424/488

(57) ABSTRACT

The present invention provides an oral delivery system for creatine. The creatine delivery system may additionally contain other bioactive ingredients such as nutraceuticals, botanicals, and vitamins. The delivery system comprises an ingestible matrix within which a creatine formulation and optionally one or more bioactives are substantially uniformly and completely dispersed and in which degradation of the creatine and other bioactives is minimised or eliminated. The invention also provides methods of preparing and using the delivery system.

### THE FIELD OF THE INVENTION

The present invention pertains to the field of oral delivery systems, in particular to an oral delivery system for creatine formulations with or without other bioactive ingredients.

### BACKGROUND OF THE INVENTION

Creatine, also known as N-(aminoiminomethyl)-N-methylglycine, methylglycoamine or N-methyl-guanido acetic acid, or n-methyl-n-guanyl glycine is widely distributed in the tissues of the body most notably in muscle, neural and reproductive tissues (Walker J. B., Creatine: Biosynthesis, regulation, and function; Adv. Enzymology and Related Areas of Molecular Biology (1979) 50: 177-242). Essentially, creatine is used biologically for the regeneration of ATP from ADP. Adenosine triphosphate (ATP) is the immediate source of energy for muscle contraction and neural activity. However the amount of ATP in muscle fibre and neural tissue is relatively small and is utilised quickly during normal activity and even faster during exercise. Therefore, a backup supply of readily available energy to be used when ATP is diminished is a biological necessity. Creatine stored as phospho-creatine serves as a reservoir of high potential phosphoryl groups that are easily transferred to ADP by a phospho-kinase reaction to form ATP. In the process of regenerating ATP phosphocreatine is used, and the creatine moiety of the molecule is spontaneously and irreversibly converted to its anhydride form, creatinine. Because creatine is irreversibly used, the body must either produce creatine biochemically or secure an outside source to supply the body with needed creatine.

It is known that the oral ingestion of creatine will add to the whole body creatine pool, wherein the ingestion of 20-30 g creatine per day for several days can lead to a greater than 20% increase in the human skeletal muscle total creatine content. Above a minimum plasma concentration creatine enters the muscle fibres, accumulates and stays, as phospho-creatine, for several weeks. Thus, the strategy behind creatine supplementation is to consume the nutrient to capacity and then to take in only amounts sufficient to maintain full storage. This creatine loading phase dosage is estimated from the total creatine storage capacity of a person's body, which is in turn directly, related to muscle mass, weight and exercise level. Recommended loading dosages, according to current literature values, range from approx. 12-25 g/day. This total dosage value is usually divided into 3 or 4 dosages (Sahelian et al., In: Creatine: Nature's Muscle Builder, 1997). Maintenance doses are determined using the same factors listed above and normally range from 4-15 g/day.

However, although muscle tissue contains approximately 0.5% creatine by weight, the cooking process degrades most of the bioavailable creatine in meat. Furthermore, creatine is not well absorbed from the gastrointestinal (GI) tract, which has been estimated to have a 1 to 14 percent absorption rate. Thus, current products require large amounts of creatine to be administered to be effective, typically 5 grams or more. High levels of creatine dosing result in certain side effects. In one survey 38% of men and 25% of women indicated they experienced serious side effects. The most common complaints were diarrhea and flatulence. The incidence of side effects increases dramatically with large dosages, (for example when greater than 120 g is consumed) or by taking creatine on an empty stomach.

Furthermore, under acidic conditions creatine is susceptible to cyclization and will form creatinine. In acidic aqueous solutions, the formation of creatinine from creatine is nearly quantitative and irreversible (Cannan, Shore, Biochem. J. 22, 924 (1928).) Therefore, it is apparent that creatine will convert to creatinine in the acidic environment of the stomach. Once creatinine is formed, any further biological use of ingested creatine will be precluded.

Promoted as a means of increasing lean muscle mass, strength and energy in addition to its potential benefit in a number of diseases (see PCT Application No. WO 02/22135; U.S. Pat. Nos. 6,242,490 and 5,576,316), the chemical compound creatine monohydrate is readily available today as a nutritional supplement in various forms such as powder, serum, liquid, and chewable. All these forms however, suffer various draw-backs such as absorbability, cyclization, ease of use, unpleasant mouthfeel, bitter taste and reported side effects such as bloating, cramps, diarrhea, nausea with dosages over five grams.

Attempts to improve the oral administration of creatine have been discussed in several patents. A number of patents disclose various combinations of creatine with different compounds in order to increase its bioavailability and to decrease some of the side effects (see U.S. Pat. Nos. 5,968;900; 5,925,378; 6,172,114; 6,139,339 and 5,773,473). However, these compounds are available in conventional pharmaceutical formats and are still susceptible to cyclization as well as a loss in organoleptic appeal.

U.S. Pat. No. 6,114,379 discloses creatine chelates that are protected from cyclization in the acid conditions of the stomach. While the chelate may be incorporated into any known pharmaceutical formats, organoleptic considerations were not addressed in the patent.

U.S. Pat. No. 5,397,786 discloses a drink for the treatment and prevention of the loss of essential electrolytes due to fluid loss. This patent teaches that creatine, B vitamins, pantothenic acid and choline are energy enhancers. Additionally, this invention provides for the addition of numerous salts such as MgCO₃, CaCO₃ and magnesium aspartate as supplements containing essential nutrients. Although the necessity of these elements in a healthy metabolism was recognized, the use of ionic salts is largely ineffective because most of the ingested elements are lost in the acidic environment of the stomach.

U.S. Pat. No. 6,274,161 relates to compositions wherein creatine is incorporated into known edible viscous liquid or semi-liquid, or solid supporting matrices that prevent the settling of creatine.

A number of patents also disclose various delivery systems for the administration of creatine or other bioactive ingredients. These range from hard confectionery compositions (see U.S. Pat. No. 6,242,019), a caramel delivery system (PCT Application No. WO 01/70238), chewable delivery systems (see U.S. Pat. Nos. 4,778,676; 4,882,153; 5,928,664; and 6,387,381 and PCT Application No. WO99/26491). Each of these systems is limited by its caloric content, its reaction with creatine, its inability to minimise the degradation of creatine and drawbacks with respect to the organoleptic appeal of the final product.

Therefore, it would be desirable to provide an improved delivery system that can enhance the absorption of creatine while minimizing the degradation of creatine. Furthermore, it would also be desirable to provide a delivery system that will overcome other side effects normally associated with the consumption of creatine.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide an oral delivery system for creatine and optionally other bioactive ingredients.

In accordance with one aspect of the present invention, there is provided an oral delivery system for creatine formulations comprising: (a) one or more sources of creatine, and (b) a matrix comprising: (i) a sugar component comprising one or more sugars, sugar syrups, sugar alcohols or a combination thereof; (ii) one or more low set temperature carbohydrates or a modified version thereof; (iii) a hydrocolloid component comprising one or more hydrocolloids; (iv) a solvent component comprising one or more polyhydric alcohols; (v) one or more sources of mono- or divalent cations, and (vi) one or more sources of water, wherein said creatine is substantially uniformly dispersed in said matrix and wherein said delivery system is a semi-solid at room temperature and has a final moisture content of between about 15% and about 30% by weight and a water activity of less than about 0.7.

In accordance with another aspect of the present invention, there is provided a method of making a delivery system for creatine formulations comprising: (a) preparing a blend of one or more carbohydrate, one or more hydrocolloid, one or more sugar, sugar alcohol or sugar syrup, or a combination thereof, one or more sources of mono- or divalent cations, and water; (b) heating said blend to a temperature of less than 100C.; (c) maintaining said blend at a temperature of less than 100C.; (d) adjusting the moisture content of the blend to between about 15% and about 30% by weight; (e) preparing a solution of one or more creatine compound or analogue thereof in a solvent component comprising one or more polyhydric alcohols at or below a temperature of 70° C.; (f) combining said blend and said solution of creatine at a temperature of 70° C. or below to form a matrix whereby the creatine is substantially uniformly dispersed throughout said matrix, and (g) forming said matrix into shapes, wherein the delivery system has a final moisture content between about 15% and about 30% and a water activity of less that about 0.7.

In accordance with still another aspect of the present invention, there is provided a use of a delivery system for creatine formulations for oral administration of creatine to an animal in need thereof.

Various other objects and advantages of the present invention will become apparent from the detailed description of the invention.

### BRIEF DESCRIPTION OF THE FIGURES

FIG. 1 demonstrates the enhanced uptake of creatine into the blood following administration of jujubes prepared according to Example 2 to humans.

FIG. 2 demonstrates serum concentrations of creatine following administration of the delivery system containing varying creatine chelate and/or creatine monohydrate formulations.

### DETAILED DESCRIPTION OF THE INVENTION

Unless defined otherwise, all technical and scientific terms used herein have the same meaning as commonly understood by one of ordinary skill in the art to which this invention pertains. As used herein, percentage values (%) represent the weight percentages of the total weight of the delivery system.

The term “animal” as used herein includes, but is not limited to, mammals including humans, birds and reptiles.

The terms “bioactive ingredient,” “bioactive agent” and “bioactive” as used interchangeably herein include physiologically or pharmacologically active substances intended for use in the treatment, prevention, diagnosis, cure or mitigation of disease or illness, or substances that provide some degree of nutritional or therapeutic benefit to an animal when consumed. Non-limiting examples include drugs, botanical extracts, enzymes, hormones, proteins, polypeptides, antigens, nutritional supplements such as fatty acids, antioxidants, vitamins, minerals, and other pharmaceutically or therapeutically useful compounds.

The Delivery System

The delivery system according to the present invention comprises an ingestible matrix within which a creatine formulation and optionally one or more bioactives are substantially uniformly and completely dispersed and in which degradation of the creatine and other bioactives is minimised or eliminated.

Typically, the delivery system comprises one or more sources of creatine substantially uniformly dispersed within a matrix which comprises 1) one or more carbohydrates that exhibit good moisture binding and low gelatinisation temperature; 2) a sugar component comprising one or more sugars, sugar syrups and/or sugar alcohols; 3) a hydrocolloid component; 4) a solvent component comprising one or more polyhydric alcohols; 5) one or more sources of mono or divalent cations; and 6) one or more sources of water. The use of one or more carbohydrates and a hydrocolloid component in amounts within the ranges indicated below results in a matrix that readily retains the solvent and thereby prevents separation of the solvent from other components of the matrix. The delivery system can further optionally. comprise one or more additional bioactive ingredients. Additives such as natural or artificial flavourings, colourings, or other active ingredients such as acidulants, buffers, and sweeteners can be included in conventional amounts in the matrix.

Due to the substantially uniform and complete dispersion of the creatine and other bioactives within the matrix, the delivery system is suitable for division into sub-units. For example, if a single unit of the delivery system is divided into three subunits, each subunit will contain a third of the dose of the original unit. Such division would not be possible with other delivery systems in which the bioactive components are not evenly dispersed.

The delivery system according to the present invention is suitable for administration to both human and non-human animals. One skilled in the art will appreciate that the delivery system can be formulated differently according to the type of animal to whch it is to be administered. For example, for administration to an animal such as a cat or a dog, meat or fish-based flavours may be added. For administration to a human, the delivery system may be formulated for example as a confectionery.

In accordance with the present invention, degradation of the creatine and/or bioactives during the process of preparing the matrix is less than about 20%. In one embodiment, degradation of the creatine and/or bioactives during preparation of the matrix is less than about 15%. In another embodiment, degradation during preparation is less than about 10%. In another embodiment, degradation during preparation is less than about 5%. In another embodiment, degradation during preparation is less than about 2%. In another embodiment, degradation during preparation is less than about 1%.

Furthermore, degradation of the creatine and bioactives dispersed within the matrix is minimised or eliminated during storage of the final delivery system under normal storage conditions (i.e. at temperatures of 30° C. or below). In accordance with the present invention, degradation of the creatine and/or bioactives during storage of the delivery system under normal conditions is less than about 20%. In one embodiment, degradation of the creatine and/or bioactives during storage is less than about 15%. In another embodiment, degradation during storage is less than about 10%. In other embodiments, degradation during storage is less than about 5%, 2% or 1%.

Minimisation or elimination of the degradation of creatine and other bioactives in the delivery system is achieved through a number of factors. For example, the use of relatively low temperatures in the preparation of the matrix when compared to typical manufacturing procedures for confectioneries ensures that the compounds are not degraded by excessive heat. In accordance with the present invention, the delivery system is prepared at a temperature of 100° C. or less. In one embodiment of the present invention, the delivery system is prepared at or below a temperature of 80° C. In another embodiment, the delivery system is prepared at or below a temperature of 75° C. These low temperatures can be employed in the preparation of the delivery system because the matrix is formulated to remain flowable at temperatures at or above 45° C. In one embodiment of the invention, the matrix remains flowable at or above 35° C.

In addition, the delivery system maintains a low interaction with water during and after preparation of the matrix. In accordance with the present invention, the final moisture content of the delivery system is between about 10% and about 30% and the water activity (a_{w}) is below about 0.7. In one embodiment of the present invention the final moisture content of the delivery system is between about 13% and about 25%. In another embodiment, the moisture content is between about 13% and about 20%. In other embodiments, the moisture content is between about 15% and about 18%, or between about 15% and about 16%. In another embodiment of the invention, the water activity of the final delivery system is below about 0.6. In another embodiment, the water activity is below about 0.55. In other embodiments, it is between about 0.45 and about 0.55, between about 0.5 and about 0.55, or between about 0.47 and about 0.52.

Acidic pH is known in the art to promote degradation of creatine and other bioactives. In accordance with the present invention, therefore, the delivery system has a final pH that is neutral to mildly basic. By neutral to mildly basic pH it is meant that the final pH of the delivery system is between about 6.0 and about 7.5. In one embodiment of the present invention, the delivery system has a final pH between about 6.2 and about 7.3. In another embodiment, the final pH of the delivery system is between about 6.5 and about 7.2.

In its final form, the delivery system of the present invention is a semi-solid, intermediate moisture system, having some properties clearly identified with those of jellies and some properties that are similar to the jujube variety of confectioneries. The matrix of the delivery system, therefore, is formulated to be semi-solid at normal room temperature. In the event, however, that the matrix liquefies due to exposure to elevated temperatures, the formulation of the matrix is such that no phase separation of the components occurs and the matrix can be readily re-solidified by cooling (for example, by cooling to temperatures of around 4° C.). The reformed product maintains the substantially uniform dispersion of the bioactives contained therein. In one embodiment of the present invention, the delivery system is formulated such that the matrix is a semi-solid at temperatures at or below about 40° C. In another embodiment, the delivery system is a semi-solid at or below about 35° C. In other embodiments, the delivery system is a semi-solid at or below about 30° C. or about 25° C.

The delivery system is especially suited for oral administration due to its palatability. Additionally, due to its highly portable format, the delivery system is simple and convenient to administer and to consume for both humans and other animals.

It will be readily apparent to one skilled in the art that new formulations of carbohydrate and hydrocolloid or modifications or substitutes thereof are being developed within the food industry. The present invention therefore contemplates the use of such new formulations to prepare the matrix of the present invention provided that the final properties of the delivery system are maintained, i.e. substantially uniform and complete dispersion of the creatine and bioactives, minimisation of the degradation of the creatine and bioactives and a final moisture content for the delivery system of between about 10% and about 30% and a water activity below about 0.7. For example, a whey-based polymer has recently been developed that acts as a gelling agent (Dairy Management Inc™). The polymer mimics gelatine functionality and forms strong gels at room temperature that exhibit large deformation without fracture and may be suitable for use in the matrix in accordance with the present invention.

The texture, physical attributes, form and shape of the matrix as described. below, can be varied by altering the ratio of ingredients within the given ranges using the methods described herein or by methods familiar to a worker skilled in the art.

One skilled in the art will appreciate that specific selections of the possible components provided below, must be safe for animal consumption. Components for inclusion in the delivery system are, therefore, substances that are generally regarded as safe (GRAS) and/or meet regulatory standards, such as those of the Codex Alimentarus. Examples falling within the general descriptions provided below that are significantly toxic or cause other types of significant harm to animal health are explicitly excluded from the description of the invention.

1. The Matrix

1.1 Carbohydrate

The carbohydrate component of the matrix typically performs the functions of water binding and gelation and contributes to the overall texture and body of the final delivery system. The carbohydrate contributes to the structural integrity of the matrix and its low set temperature. The carbohydrate can also provide heat stability to the finished product as well as the ability to bind a limited quantity of fats/oils if required.

The carbohydrate to be included in the matrix is selected for its ability to fully hydrate and develop its viscosity in the presence of the other matrix-forming components at a temperature below 100° C. The selected carbohydrate should thus be capable of dispersing without clumping in a sugar syrup or in water, and of becoming fully hydrated with or without heating either in the presence of a sugar syrup or another source of water. While the majority of carbohydrates hydrate upon heating, certain starches, which are commercially available and are known in the art as “cold set” or pre-gelatinised” starches are capable of hydrating at room temperature and are suitable for use in the matrix according to the present invention.

In accordance with the present invention, therefore, the selected carbohydrate is capable of hydrating and developing its viscosity at a temperature below 100° C. In one embodiment, the carbohydrate is capable of hydrating at or below 70° C. In another embodiment, the carbohydrate is capable of hydrating at or below 50° C. In other embodiments, the carbohydrate is capable of hydrating at or below 40° C., 35° C. or 25° C.

Furthermore, the selected carbohydrate should allow the final matrix to remain in a free-flowing state at a sufficiently low temperature for addition of creatine and other bioactive ingredients without significant degradation of these compounds. In accordance with the present invention, therefore, the carbohydrate remains free-flowing at or below 100° C. In one embodiment of the present invention, the carbohydrate remains free-flowing between about 35° C. and about 85° C. In another embodiment, the carbohydrate remains free-flowing between about 45° C. and about 70° C.

The viscosity development of the selected carbohydrate should allow for sufficient ease of mechanical handling and pumping during production as well as allowing sufficient time to incorporate all the ingredients and to mould the final product before it sets. As is known in the art, some carbohydrates develop their viscosity upon heating, whereas others develop viscosity upon cooling. Both types of carbohydrates are considered to be suitable for use in the matrix of the present invention. In one embodiment, the selected carbohydrate will develop its viscosity upon cooling. In another embodiment, the viscosity of the carbohydrate will develop completely after deposition or filling.

Examples of a type of carbohydrate suitable for use in the matrix of the present invention are starches and other amylaceous ingredients that have been modified such that they have a low set temperature. An amylaceous ingredient as used herein refers to a food-stuff that contains a preponderance of starch and/or starch-like material. Examples of amylaceous ingredients include cereal grains and meals or flours obtained upon grinding cereal grains such as corn, oats, wheat, milo, barley, rice, as well as the various milling by-products of these cereal grains such as wheat feed flour, wheat middlings, mixed feed, wheat shorts, wheat red dog, oat groats, hominy feed, and other such material. Other sources of amylaceous ingredients include tuberous foodstuffs, such as potatoes, tapioca, and the like.

Suitable starches are typically modified starches and include those derived from a natural source, such as those obtained from various plant species. Examples of plant sources of starch include, but are not limited to, corn, waxy corn, wheat, rice, tapioca, potato, pea and other sources known in the art. Modified starches are known in the art and the term generally refers to starch that has been physically or chemically altered to improve its functional characteristics. Suitable modified starches include, but are not limited to, pre-gelatinised starches, low viscosity starches (such as dextrins, acid-modified starches, oxidized starches and enzyme modified starches), derivatised starches, stabilised starches (such as starch esters and starch ethers), cross-linked starches, starch sugars (such as glucose syrup, dextrose and isoglucose) and starches that have been submitted to a combination of treatments (such as cross-linking and gelatinisation) and mixtures thereof. The carbohydrate may also be a synthetic starch substitute provided that it meets the criteria outlined herein.

In one embodiment of the present invention, the carbohydrate is a modified starch. In another embodiment, the modified starch is a modified cornstarch. Examples of commercially available cornstarches include Soft-Set® and MiraQuick® (A. E. Staley Manufacturing Co.).

In accordance with the present invention, the amount of carbohydrate included in the matrix ranges from about 1% to about 15% by weight. The selection of the actual amount of carbohydrate from within this range to be included in the matrix will be dependent upon the desired texture of the final product and determination of this amount is considered to be within the ordinary skills of a worker in the art. Thus, for example, for a delivery system with a final texture similar to gum drop confectionery, the amount of carbohydrate will be between about 9% and about 14%. In one embodiment of the present invention, the amount of carbohydrate included in the matrix is between about 2% and about 15%. In other embodiments, the amount of carbohydrate is between about 2% and 10%, between about 2% and about 8%, between about 2% and about 5%, or between about 2% and about 3%.

1.2 Sugar Component

Sugar is generally used in a confection primarily for sweetness; however, it is known in the art that sugar can also play an important role in the physical properties of a matrix, such as crystallinity, gel strength, bodying/texture, humectancy, and water activity.

The sugar component of the matrix comprises one or more sugars, sugar syrups, sugar alcohols and/or sugar alcohol solids. Examples include, but are not limited to, sugars such as sucrose, glucose, xylose, ribose, maltose, galactose, dextrose, and fructose; syrups such as corn syrups, hydrogenated glucose syrups, high fructose corn syrups; polydextrose; and sugar alcohols such as isomalt, maltitol, sorbitol, lactitol and mannitol. One skilled in the art will appreciate that if a sugar or sugar alcohol solid is used in the matrix, it should be first dissolved, for example, by heating in water or in another syrup, prior to being added to the mixture.

When the sugar used to prepare the matrix is dextrose, it is generally provided in the form of a corn syrup. Corn syrups are prepared by hydrolysis of starch and are characterized by dextrose equivalent (D.E.) values such that they are classified as low, medium or high D.E. syrups, with high D.E. syrups having a high concentration of dextrose and low D.E. syrups having a low concentration of dextrose. In one embodiment of the present invention, the sugar component used in the preparation of the matrix comprises a corn syrup. In another embodiment, the matrix comprises a corn syrup that exhibits a D.E. of between 20 D.E. and 99 D.E. In other embodiments, the matrix comprises a “high” DE corn syrup with a D.E. of between 40 and 70, or with a D.E. of between 62 and 65.

Various corn syrups are commercially available. For example, 62 D.E. 1600 Corn Syrup (Casco Inc./Canada Starch Operating Co. Inc.), SWEETOSE 4300 corn syrup (a 63 D.E. corn syrup; A. E. Staley Manufacturing Company; Decatur, Ill.) and Clearsweet® 63/43 IX corn syrup (a 63 D.E. corn syrup; Cargill/North America Sweeteners).

One skilled in the art will appreciate that the total amount of sugar in the matrix will vary depending upon the combination of sugar sources used. For example, when sugar syrups are used, lower viscosity sugar syrups will produce a matrix with less body and lower rigidity. The total amount of sugar present in the matrix is about 20% to about 60% by weight. In one embodiment of the present invention, the total amount of sugar in the matrix is between about 35% and about 55% by weight In another embodiment, the total amount of sugar in the matrix is between about 40% and about 50% by weight.

1.3 Hydrocolloid Component

The matrix according to the present invention further comprises one or more hydrocolloid. Hydrocolloids are hydrophilic polymers of vegetable, animal, microbial or synthetic origin, naturally present or added to aqueous foodstuffs for a variety of reasons due to their unique textural, structural and functional properties. In general, they are used for their thickening and/or gelling properties as well as their water binding and organoleptic properties. Hydrocolloids can also be used to improve and/or stabilize the texture of a food product while inhibiting crystallization.

Examples of hydrocolloids include, but are not limited to, tragacanth, guar gum, acacia gum, karaya gum, locust bean gum, xanthan gum, agar, pectin, gelatine, carageenan, gellan, alginate, or a combination thereof. The use of hydrocolloids is well-known in the art and many hydrocolloids for use in products for human or animal consumption are available commercially, for example, Type B gelatine from Leiner Davis, Kelcogel® Gellan Gum manufactured by CP Kelco and a range of Ticagel® hydrocolloids from Tic Gums.

One skilled in the art will appreciate that the selection of the hydrocolloid to be used in the matrix will depend on the pH of the matrix, the interaction of the hydrocolloid with the carbohydrate component of the matrix and the particular texture and consistency required for the final product. The type of hydrocolloid used will also affect the set temperature of the matrix. For example, the use of a gelatine/gellan mixture provides a set temperature around 35° C., whereas the use of carageenan or locust bean gum will result in a set temperature closer to 60° C. Thus, the choice of hydrocolloid for use in the matrix is also dependent upon the properties of the bioactive ingredient(s) to be incorporated into the delivery system. Bioactives that are unstable at higher temperatures will require the selection of a hydrocolloid or mixture of hydrocolloids that have a low set temperature, whereas bioactives that are more stable can be used with hydrocolloids having a higher set temperature. Selection of an appropriate hydrocolloid is considered to be within the ordinary skills of a worker in the art.

In one embodiment of the present invention, the hydrocolloid is gelatine. The term “gelatine” refers to a heterogeneous mixture of water-soluble proteins of high average molecular weight derived from the collagen-containing parts of animals, such as skin, bone and ossein by hydrolytic action, usually either acid hydrolysis or alkaline hydrolysis. Different types of gelatine can be prepared by altering the process parameters. Gelatine is defined generally using a “Bloom value” which indicates the strength of the gel formed under certain circumstances using the gelatine. In the preparation of confectionery, when a harder gel is desired, gelatine having a higher Bloom value is used. Conversely, when the final product is required to be more flowing, gelatine having a lower Bloom value is used. One skilled in the art will appreciate that the water holding capacity of gelatine alone is lower than that of a combination of gelatine with another hydrocolloid, such as gellan, and may necessitate the use of a higher amount of gelatine to achieve the desired gelation/texture of the matrix. When the hydrocolloid in the matrix of the present invention comprises gelatine, the Bloom value (BL) is generally about 100 to 260 BL. In one embodiment, the Bloom value is about 250 BL. In another embodiment, a mixture of gelatines with different Bloom values is used.

In an alternative embodiment of the present invention, the hydrocolloid is a mix of gelatine and gellan. When this combination of hydrocolloids is used, gelatine is mixed with gellan in a ratio of between about 15:1 to about 40:1 in order to ensure the cohesive structure of the delivery system. In one embodiment of the present invention, a ratio of about 20:1 to about 35:1 of gelatine:gellan is used.

The total amount of hydrocolloid incorporated into the matrix is generally between about 0.1% and about 7.0% by weight. In one embodiment, the total amount of hydrocolloid in the matrix is between about 0.5% and about 5.0% by weight. In another embodiment, the total amount is between about 1.0% and about 4.0%. In other embodiments, it is between about 1.0% and about 3.0% or between about 1.0% and about 2.0%.

1.4 Solvent Component

The primary role of the solvent component of the matrix is to dissolve or disperse the creatine and other bioactives to allow for substantially uniform and complete incorporation of these ingredients into the matrix. The solvent also provides for improved flow characteristics of the mixture and functions somewhat as a humectant. In accordance with the present invention, the creatine and optional other bioactive ingredients are added to the solvent component prior to combining with the remaining components of the matrix.

The solvent used in the preparation of the matrix is typically colourless, non-volatile with no strong odour or flavour and is substantially miscible with water and/or alcohols. In accordance with the present invention, the solvent component can be one or more polyhydric alcohol. The term “polyhydric” as used herein means that the compound contains two or more hydroxyl groups. Examples of polyhydric alcohols include, but are not limited to, glycerol and/or its lower alkyl ester derivatives, sorbitol, propylene glycol, and short chain polyalkylene glycols, such as polyethylene glycol, and mixtures thereof. In one embodiment of the present invention, the solvent component comprises glycerol. In another embodiment, the solvent component comprises glycerol and a short chain polyalkylene glycol.

One skilled in the art will understand that the amount of the solvent component incorporated into the matrix will be dependent on the solubility of the bioactive ingredient(s) being incorporated into the delivery system. Typically, the delivery system according to the present invention contains about 5% to about 35% by weight of the solvent component. In one embodiment, the delivery system contains about 20% to about 30% of the solvent component.

1.5 Mono- or Divalent Cations

The matrix also comprises one or more sources of mono- and/or divalent cations in order to allow proper gelation of the matrix. The mono- and/or divalent cations can be provided in the form of a creatine chelate or they may be added separately to the matrix during preparation. Suitable sources of mono- and divalent cations for incorporation into food products are known in the art and are commercially available. Examples include mono- or divalent salts such as sodium, potassium or calcium chloride or potassium citrate. In one embodiment of the present invention, potassium citrate is added to the matrix as a source of monovalent cations.

When an additional source of mono- or divalent cations is required and is provided in the form of a mono- or divalent salt, then it is typically added to the matrix in an amount between about 1% and about 5% by weight. In one embodiment it is added in an amount between about 1% and about 3%. In another embodiment, it is added in an amount between about 1.2% and about 2.5%.

6 Water

As indicated above, the delivery system according to the present invention has a final moisture content between about 10% and about 30% and a water activity below about 0.7. It will be readily apparent to one skilled in the art that the appropriate amount of water may be provided by one or more of the various components of the system, for example, a sugar syrup, a hydrated starch or a hydrated hydrocolloid, or additional water may need to be added separately. Additional water can be provided alone or as a solution containing other additives, for example, as a buffer solution or as a solution containing a sweetener, flavouring or colouring. The total amount of water from the one or more sources will be sufficient to provide the final delivery system with a moisture content and water activity within the ranges indicated above.

1.7 Other Additives

The matrix can optionally contain other additives such as sweeteners, flavourings, colourings, modified vegetable gums or celluloses, or a combination thereof. It will be readily apparent that additives for inclusion in the matrix should be selected such that they do not affect the properties of the matrix, do not exhibit substantial reactivity with the creatine or bioactives in the matrix, and are stable during preparation of the matrix.

The sweetener can be selected from a wide variety of suitable materials known in the art. Representative, but non-limiting, examples of sweeteners include xylose, ribose, sucrose, mannose, galactose, fructose, dextrose, maltose, partially hydrolyzed starch, lactose, maltodextrins, hydrogenated starch hydrolysate and mixtures thereof. In addition to these sweeteners, polyhydric alcohols such as sorbitol, mannitol, xylitol, and the like may also be incorporated. Alternatively, one or more artificial sweeteners can be used, for example, sucrose derivatives (such as Sucralose), amino acid based sweeteners, dipeptide sweeteners, saccharin and salts thereof, acesulfame salts (such as acesulfame potassium), cyclamates, steviosides, dihydrochalcone compounds, thaumatin (talin), glycyrrhizin, aspartame, neotame, alitame, and mixtures thereof.

When an additional sweetener is used, it can be used in amounts as low as 0.01% by weight. The actual amount of sweetener required will be dependent on the type of sweetener selected and on the desired sweetness of the final product. Amounts of various sweeteners to be added to food products are well known in the art. The total amount of the sugar component, which forms a structural part of the matrix, and additional sweetener(s) in the matrix, however, is less than 60% by weight.

Suitable flavourings that can be added to the delivery system are known in the art and include, both synthetic flavour oils and oils derived from various sources, such as plants, leaves, flowers, fruits, nuts, and the like.

Representative flavour oils include spearmint oil, peppermint oil, cinnamon oil, and oil of wintergreen (methylsalicylate). Other useful oils include, for example, artificial, natural or synthetic fruit flavors such as citrus oils including lemon, orange, grape, lime and grapefruit, and fruit essences including apple, strawberry, cherry, pineapple, banana, raspberry and others that are familiar to a worker skilled in the art.

The amount of flavouring agent employed is normally a matter of preference subject to such factors as concentration/dilution of the flavour stock, flavour type, base type and strength desired. In general, amounts of about 0.01% to about 5.0% by weight of a final product are useful. In one embodiment of the present invention, a flavouring agent is included in the matrix in amounts of about 0.02% to about 3%. In another embodiment, the flavouring agent is added in amounts of about 0.03% to about 1.5%.

Colourings suitable for use in foodstuffs are well known in the art and can be optionally included in the matrix to add aesthetic appeal. A wide variety of suitable food colourings are available commercially, for example, from Warner Jenkins, St. Louis, Mo. Where a synthetic colouring agent is used in the matrix, the amount ranges from about 0.01% to about 2% by weight. In one embodiment of the present invention, a synthetic colouring agent is added to the matrix in an amount between about 0.03% to about 1% by weight. A worker skilled in the art will appreciate that when a colouring agent derived from a natural source is used in the matrix, an increased amount of the colouring agent is generally required to achieve the same effect as a synthetic colouring agent.

The present invention also contemplates that modified vegetable gums or celluloses may be included in the matrix in order to improve the texture, lubricity and/or elasticity of the matrix. These compounds can be used, for example, to increase the viscosity of the delivery system if it is. warmed, thus reducing potential melting and lessening water activity which will help to improve the stability of the system in the event it is left in an excessively hot environment. The use of modified vegetable gums or celluloses, such as methylcellulose and hydroxypropyl methylcellulose, for this purpose is well known in the food industry. For example, a range of modified vegetable gums or celluloses known as Methogel Food Gums are manufactured by Dow Chemical Company and are typically included in caramel and nougat type confectioneries in amounts between about 0.3% and 0.5% by weight.

2. Creatine

The delivery system of the present invention comprises creatine. The term “creatine” as used herein encompasses various forms of creatine, or N-aminoiminomethyl-N-methylglycine, currently available for animal consumption as well as those that will become available in the future, including creatine hydrates, creatine salts, creatine chelates, and prodrugs and protected or modified forms of creatine that are metabolised in the body to form creatine. For example, the present invention contemplates the use of creatine monohydrate and other creatine hydrates, creatine salts such as creatine citrate, creatine pyruvate, creatine phosphate and other suitable salts. Creatine chelates are also contemplated in the present invention, such as those described in U.S. Pat. No. 6,114,379, which are commercially available through Albion Laboratories, Inc. Prodrugs that metabolise to yield creatine in the body are also contemplated, such as glycocyamine (guanidoacetic acid), as are protected and modified forms of creatine that can be metabolised in the body, for example, those described in International Patent Application WO02/22135. Finally, analogues, derivatives, optical isomers and biologically active salts or esters of creatine that provide the same pharmaceutical results are also contemplated. Methods of preparing creatine for use in the present invention are known in the art. Additionally, commercial sources of creatine are also readily available.

It will be understood that the amount of creatine to be included in the delivery system will be dependent upon the particular animal for which the final product is prepared. Suitable amounts of creatine for human and animal applications are known in the art. The amount of creatine present in the delivery system will also be dependent on whether other bioactives are to be included in the system. In the absence of other bioactives, the total amount of creatine in the delivery system, however, will be less than or equal to 25% by weight. In one embodiment of the present invention, the total amount of creatine in the delivery system is less than or equal to 20% by weight. In another embodiment, the total amount of creatine in the delivery system is less than or equal to 18% by weight. In another embodiment, the total amount of creatine in the delivery system is less than or equal to 17% by weight.

3. Bioactive Ingredients

The delivery system according to the present invention may further comprise one or more bioactive ingredients. Selection of appropriate bioactive agents for incorporation into the delivery system for administration to a given animal is considered to be within the ordinary skills of a worker in the art and it is understood that bioactive agents suitable for administration to humans may differ from those suitable for other animals. Furthermore, it will be apparent that inappropriate combinations of bioactive agents, for example, those that interact with each other, or those that interfere with the uptake of creatine, such as caffeine, theobromine and the like, should not be included in the delivery system. Bioactive ingredients that cause the acidification of the matrix are not considered to be appropriate for incorporation in to the delivery system according to the present invention, as these may lead to degradation of the creatine under certain conditions.

Bioactive ingredients are incorporated into the delivery system at levels sufficient to affect the structure or function of the body when taken regularly. Such levels are known in the art or can readily be determined by a skilled technician. It is understood that the total daily intake may be based on administration of one unit of the delivery system, or it may be based on administration of more than one unit. The amount of bioactive ingredients in the final product will thus vary depending on the format of the units and the number to be administered daily.

Typically, the total amount of bioactive ingredients including the one or more sources of creatine constitute less than about 25% by weight of the delivery system. In one embodiment of the present invention, the total amount of bioactive ingredients constitutes between about 5% and about 20% by weight of the delivery system. In another embodiment, the total amount of bioactive ingredients constitutes between about 5% and about 20% by weight of the delivery system.

3.1 Drugs

One or more of the bioactive ingredients incorporated into the delivery system may be a drug. The term “drug” as used herein refers to a substance that exerts a therapeutic effect on an animal. Examples of suitable drugs for use in the present invention include, but are not limited to, anti-hypertensive drugs, vasoconstrictors, sedatives, antibiotics, antihistamines, decongestants, expectorants, and anti-nauseants.

3.2 Nutritional Supplements

One or more of the bioactive ingredients included in the delivery system can be a nutritional supplement. The term “nutritional supplement” as used herein refers to a substance that exerts a physiological effect on an animal. Illustrative, but non-limiting examples of nutritional supplements suitable for use with the delivery system according to the present invention include, probiotic bacteria, prebiotics, vitamins, enzymes, co-enzymes, cofactors, antioxidants, minerals and mineral salts, amino-acids and amino acid derivatives, peptides, proteins, gums, carbohydrates, phytochemicals, dextroses, phospholipids, other trace nutrients, oxygenators, brain-stimulating substances, energy providers, metabolic intermediates, hormones, botanical extracts, fatty acids, oat beta-glucan or other functional fibres, carnitine, bicarbonate, citrate, or combinations thereof.

Formulations of nutritional supplements may be incorporated into the delivery system, for example, L-arginine, co-enzyme Q10, human growth hormone, glutathione precursors, N,N dimethylglycine, chromium-niacin complex with hydroxycitric acid and devil's club, glucosamine, multi-vitamins and minerals, methoxyisoflavones, chitosan, methylsulphonylmethane, and conjugated linoleic acids.

Probiotic microorganisms in the form of live microbial nutritional supplements and which are recognized as conferring a beneficial effect on an animal can be incorporated into the delivery system. Probiotic microorganisms are microorganisms which beneficially affect a host by improving its intestinal microbial balance (see, for example, Fuller, R; 1989; J Applied Bacteriology, 66: 365-378). Beneficial effects of probiotic microorganisms include activation of the immune system, prevention of the bacterial overgrowth by pathogens, prevention of diarrhoea and/or restoration of intestinal flora. Examples of probiotic microorganisms include, but are not limited to, Bifidobacterium (such as Bifidobacterium longum B129, Bifidobacterium longum B128, Bifidobacterium adolescentis Bad4, and Bifidobacterium lactis Bb12), Lactobacillus (such as, Lactobacillus johnsonii and Lactobacillus paracasei), Streptococcus and Saccharomyces. Typically, the microorganism is added to the matrix in a spray dried or freeze-dried form.

Many probiotic bacterial strains have been deposited under the Budapest Treaty at the Collection Nationale de Cultures de Microorganismes (CNCM), Institut Pasteur, 28 rue du Docteur Roux, 75724 Paris Cedex 15, France. For example, Lactobacillus johnsonii (NCC 533) has been deposited on 30.06.1992 under reference CNCM I-1225, Lactobacillus paracasei (NCC 2461) has been deposited on 12.01.1999 under reference CNMC I-2116, Bifidobacterium longum (B129) (NCC490) has been deposited on 15.03.1999 under reference CNCM I-2170, Bifidobacterium longum (B128) (NCC481) has been deposited on 15.03.1999 under reference CNCM I-2169, and Bifidobacterium adolescentis (Bad4) (NCC251) has been deposited on 15.03.1999 under CNCM I-2168. Bifidobacterium lactis (Bb12) may be obtained at Hanzen A/S, 10-12 Boege Alle, P.O. Box 407, DK-2970.

The amount of probiotic incorporated into the delivery system will vary according to the specific needs. Typically, the amount of lactic acid bacteria in one unit of the delivery system is between 10² and 10¹² count/gram, for example, between 10⁷ and 10¹¹ count/gram, or between 10⁸ and 10¹⁰ count/gram.

Prebiotics can be delivered alone or in combination with probiotic bacteria in the delivery system. Prebiotics comprise carbohydrates, generally oligosaccharides, and have the ability to resist hydrolysis by enzymes in the animal digestive tract and thus can reach the colon undegraded to provide a carbohydrate substance particularly suited to growth of probiotic bacteria. Oligosaccharides may be produced from glucose, galactose, xylose, maltose, sucrose, lactose, starch, xylan, hemicellulose, inulin, or a mixture thereof. Purified commercially available products such as fructooligosaccharide contain greater than about 95% solids in the form of oligosaccharides. Prebiotics often comprise a mixture of fructooligosaccharide and inulin, for example, PREBIO1® or a mixture of commercially available RAFTILOSE® and RAFTILINE® commercialized by Orafti. A prebiotic of this kind has been demonstrated to improve the response of the immune system.

Other suitable nutritional supplements include vitamins and minerals that the body is usually not capable of synthesizing and which are necessary for ensuring normal growth and/or daily body maintenance. In the context of the present invention, the vitamins can be hydrosoluble or liposoluble vitamins. Examples includes, but are not limited to, Vitamin A (axerophtol or retinol), Vitamin D, Vitamin E (alpha-tocopherol), Vitamin K, Vitamin B and/or PP (niacinamide or nicotinic acid amide) and Vitamin C (L-ascorbic acid). The dosage of vitamins in the delivery system can be adapted to specific needs. In general, one unit of the delivery system may contain a fraction of the recommended daily amount (RDA) of the desired vitamin. For example, assuming a daily consumption of five units of the delivery system, and following European RDA recommendations, Vitamin A can be used up to 160 μg typically between 70 μg and 90 μg a single unit; Vitamin C up to 12 mg typically between 5 mg and 7 mg a single unit; Vitamin E up to 2 mg typically between 0.8 mg and 1.2 mg a single unit; Vitamin D up to 1 μg typically between 0.4 μg and 0.6 μg a single unit; Vitamin B1 up to 0.28 mg typically between 0.12 mg and 0.15 mg a single unit.

Antioxidants can be delivered using the delivery system of the present invention, alone or in combination with other bioactive agents, such as glutathione, peroxidase, superoxide dismutase, catalase, co-enzyme Q10, honey tocopherols, lycopenes, beta-carotene or other carotenoids, quertin, rutin, flavonoids, catechins, anthocyanes, eleutherosides and ginsenosides. Some of these antioxidants may be found in significant amounts in plant extracts. Examples include Ginko Biloba leaves that contain Gingko flavanoids, Blueberry fruits that contains anthocyanids, Ginseng roots which contains ginsenosides, Eleutherococcus roots which contains eleutherosides. The biologically active agent may also be a phytochemical such as polyphenol, procyanidin, phenolic acid, catechin or epicatechin, isoflavone, terpene or other phytonutritive plant material.

Suitable minerals include macro-nutrients such as sodium, potassium, calcium, magnesium, phosphorus or oligo-elements such as iron, zinc, copper, selenium, chromium, iodine or a combination thereof. Macro-nutrients are known to play an essential role in complex metabolisms of the body such as in cellular cation exchange, for example, calcium is an essential constituent of the skeleton. Following EU RDA recommendations and assuming, for instance, an average daily consumption of 5 units of the delivery system. Calcium may be used in amounts of up to 160 mg, typically between 60 mg and 90 mg in a single unit.

Trace elements are minerals present in the human body in quantity of usually less than 5 g. An example of a trace element is zinc, which has antioxidant properties, helps in the synthesis of metallothionein, is an essential factor for protein synthesis and helps improve the function of the immune system. Following EU RDA recommendations and assuming a daily consumption of 5 units of the delivery system, zinc may be used in amounts of up to 3 mg per unit, typically between 1.3 mg and 1.7 mg.

Selenium is also an antioxidant and is a co-factor for glutathione peroxidase. Selenium is known to contribute to the integrity of muscles and sperm and also plays a role in hepatic metabolism. Selenium deficiencies may lead to sever cardiac, bone or neuromuscular damage. For example, following the European RDA recommendations and assuming a daily consumption of 5 units of the delivery system, Selenium may be used in amounts of up to 11 μg per unit, typically between 4 μg and 6 μg in humans.

Other nutritional supplements include amino acids, di-peptides or polypeptides or proteins or essential fatty acids. A suitable example of an amino acid is glutamine which provides fuel to gastro-intestinal and immune cells, reduces bacterial translocation and helps prevent muscle loss and improves nitrogen balance. Examples of peptides are the glycopeptides of lactic origin active in inhibiting the adhesion of the bacteria responsible for dental plaque and caries. More particularly, dental and anti-plaque caries agents of this type comprise active principle(s) selected from kappa-caseino-glycopeptides and deacylated derivatives thereof (also known as “CGMP”). Such active principles have an effectiveness on the dental plaque only after a few seconds in the mouth (see, for example, European Patent Number EP283675). Other peptides include phosphopeptides or salts thereof having anticarie properties such as those having from 5 to 30 amino acids including the sequence A-B-C-D-E where, A, B, C, D and E being independently phosphoserine, phosphothreonine, phosphotyrosine, phosphohistidine, glutamate and aspartate and compositions particularly compositions to teeth including same (see, for example, U.S. Pat. No. 5,015,628).

Other examples of polypeptides are cysteine, acetylcysteine, cysteine methionine or a combination thereof. Cysteine and its derivatives are known to aid in defence against oxidative stress and in protein synthesis.

Other nutritional supplements include functional fibres, phospholipids, enzymes known to aid digestion (such as papain, bromelain and lipases), shark cartilage extracts, Brewer's yeast, blue green algae and the like.

The nutritional supplement can be a botanical extract, such as Guarana, Gingko Biloba, Kola nut, Goldenseal, Goto Kola, Schizandra, Elderberry, St. John's Wort, Valerian and Ephedra, evening primrose oil, beta-sitosterol, cafestol, D-limonene, kabweol, nomilin, oltipraz, sulphoraphane, tangeretin, black tea, white tea, java tea, folic acid, garlic oil, fiber, green tea extract, lemon oil, mace, licorice, menthol, onion oil, orange oil, rosemary extract, milk thistle extract, Echinacea, Siberian ginseng or Panax ginseng, lemon balm, Kava Kava, matte, bilberry, soy, grapefruit, seaweed, hawthorn, lime blossom, sage, clove, basil, curcumin, taurine, wild oat herb, dandelion, gentian, aloe vera, hops, cinnamon, peppermint, grape chamomile, fennel, marshmallow, ginger, slippery elm, cardamon, coriander, anise, thyme, rehmannia, eucalyptus, menthol, kava kava, and schisandra.

4. Method of Testing Incorporation of the Bioactive Ingredient(s) into the Delivery System

One skilled in the art will appreciate that molecular interaction between the additional bioactive ingredient and the matrix may affect the physical attributes of the final product. For example, the addition of an acidic bioactive ingredient may prevent the proper gelation of the hydrocolloid component(s), and thus would require the addition of suitable buffer salts to correct the pH. A sample of the delivery system, therefore, is prepared prior to large-scale production in order to determine whether the matrix retains the desired physical properties after inclusion of the bioactive ingredient(s), as described below. In addition, analysis of creatine levels in the matrix containing bioactive ingredients may also be conducted to determine any possible degradation of the creatine.

Process for Preparing the Delivery System

In accordance with the present invention, the delivery system remains flowable at temperatures below 100° C. to allow for full dispersion and incorporation of creatine and optionally other bioactives into the matrix while minimising or preventing degradation of these compounds. Thus, although the actual methodology used to prepare the delivery system may vary depending on the individual components selected to make up the matrix, the process of preparing the matrix comprises the step of adding the creatine (and optionally other bioactives) dispersed in the solvent component to the liquid matrix at temperatures below 100° C. Various standard methods known in the confectionery manufacturing industry can be used to prepare the delivery system and selection of the appropriate method is considered to be within the ordinary skills of a worker in the art. Batch processes, such as kettle cooking, as well as continuous processes, such as direct stream injection jet cookers and indirect stream tubular heat exchangers, are suitable for preparing the delivery system.

The following description represents one general method of preparing the delivery system according to the present invention.

Briefly, a blend of the carbohydrate, hydrated hydrocolloid and the sugar component is prepared. This blend is heated to a temperature of less than 100° C., for example between 75° C. and 80° C., such that all ingredients are dissolved and the desired moisture content is achieved (i.e. 10%-30%). The temperature of the mixture is then reduced to between 50° C. and 80° C.

A solution of creatine in solvent is prepared at or below 70° C., for example below 50° C. As will be apparent to one skilled in the art, if a non-chelated form of creatine is used, then a source of mono- or divalent cation must be added to allow for proper set up of the matrix. The prepared creatine solution is added to the carbohydrate, hydrocolloid and sugar mixture prepared as indicated above. Other bioactives, flavourings and colourings may optionally be added after this step. The matrix can then be moulded, for example, using the standard Mogul process or by injection-filling of pre-formed moulds. In accordance with the present invention, the final product has a moisture level between 10% and 30%, for example between 15% and 20%, and a water activity of less than 0.7. The pH of the matrix is adjusted to be neutral to mildly alkaline (i.e. between 6.0 and about 7.5) in order to support the stability of the creatine and other bioactive ingredients contained within the delivery system. Suitable methods of adjusting the pH of food products are known in the art and include, for example, the addition of buffers, acids or bases, such as polyphosphates, sodium hydroxide or potassium hydroxide.

In its final form, the delivery system of the present invention is a semi-solid, intermediate moisture system, having some properties clearly identified with those of jellies and some properties that are similar to the jujube variety of confectioneries. The matrix of the delivery system is thus formulated to be semi-solid at normal room temperature (i.e. at temperatures between about 20° C. and about 30° C.). It will be readily apparent that depending on the components utilised in the matrix, the amount of each to be included in the matrix may need to be manipulated within the ranges indicated in order to achieve a semi-solid, intermediate moisture product. One skilled in the art of confectionery design can readily determine which component will need to be adjusted in order to achieve an end-product with these physical properties.

Similarly, it will be readily apparent to one skilled in the art that variations can be made to the described process dependent on the type and the actual amount of each component used (within the given ranges) in order to obtain the same end product. For example, if the carbohydrate component is a starch, it is known in the art that the gelatinisation temperature of the starch may be affected when certain sugars and sugar alcohols are used. If required, therefore, starch, hydrated hydrocolloid and the sugar component can be heated above 100° C. to allow full gelatinisation of the starch to occur and the desired moisture content to be reached. The temperature of the mixture can then be reduced to between 50° C. and 80° C. prior to addition of the creatine/solvent blend and optionally other bioactives, flavourings and colourings.

Similarly, the choice of hydrocolloid can affect the set up temperature of the matrix. The use of a combination of gelatine and gellan, such as a gelatine:gellan ratio of between about 20:1 and about 40:1, as the hydrocolloid, for example, results in a matrix set-up temperature of about 35° C. In contrast, the use of other hydrocolloids or combinations of other hydrocolloids with or without gelatine or gellan, can affect the set up temperature of the matrix. For example, the use of locust bean gum or carageenan results in set up temperatures of around 60° C. The choice of hydrocolloid is thus dependent on the bioactive to be incorporated into the matrix. Temperature sensitive bioactives will require a hydrocolloid or hydrocolloid mixture that provides a low set up temperature (such as the gelatine:gellan mixture described above), whereas other hydrocolloids or mixtures thereof can be used with bioactives that can tolerate higher temperatures.

The manner in which the individual components are combined may also be varied provided that the creatine is dispersed in solvent prior to addition to the remainder of the components. For example, the hydrocolloid and part of the sugar component can be mixed and heated prior to being blended with the carbohydrate and remainder of the sugar component. Alternatively, the carbohydrate and the sugar component can be mixed and heated prior to addition of the hydrated hydrocolloid. These and other variations are considered to be within the scope of the present invention.

In one embodiment of the present invention, the matrix is prepared using (a) starch; (b) gelatine:gellan as the hydrocolloid; (c) a mixture of corn syrup and fructose syrup as the sugar component, (d) a mixture glycerol and propylene glycol as the solvent component, (e) potassium citrate as a source of monovalent cations, and (f) water. The process comprises blending the glycerol and propylene glycol, adding creatine and warming the resulting blend to 65-70° C. The fructose syrup is blended with water and warmed to 60° C. The gelatine is blended with the gellan, added to the fructose syrup with constant agitation and the temperature is raised to 75° C. in order to dissolve all the components. The corn syrup is warmed to 30-35° C. and the starch and potassium citrate are blended in. The gelatine:gellan blend and the starch blend are then combined and the solution is maintained at 75-80° C. in order to reduce the moisture content to the desired solids level. Once this has been achieved, the creatine mixture is added, together with any desired colouring and flavouring. The resulting matrix is then moulded using standard procedures.

In another embodiment of the present invention, a matrix containing the same components as indicated above, is prepared by the following process. Glycerol and propylene glycol are blended together, creatine is added and the resulting solution is blended and warmed to 40° C.-60° C. The corn and fructose syrups are blended with water and heated. The dry ingredients are blended and combined with the warmed syrups. The mixture is then heated to at least 80° C. In an alternative embodiment, the blended dry ingredients are blended in with simultaneous live steam injection to reach at least 80° C. The solid content is then adjusted by addition of water to provide a final moisture content of 10% to 30%. The temperature of the syrup mixture is lowered to between 50° C. and 80° C. and the creatine blend is incorporated. Finally, any desired colouring and flavouring is added. The matrix is then injection filled into preformed packaging.

Testing the Delivery System

Once the delivery system has been prepared it is important that it is tested to ensure that it meets the desired criteria, i.e. that the creatine and other bioactives are substantially uniformly dispersed, that degradation of these compounds during the preparation of the matrix is below 20% and that the water activity of the delivery system is below 0.7.

For example, dispersion of the product can be determined by dividing a single unit of the final delivery system into several subunits and analysing the content of creatine in each subunit. Creatine levels can readily be measured by standard analytical techniques such as chromatographic techniques. In one embodiment of the present invention, the level of creatine in the final product is analysed by high performance liquid chromatography (HPLC). If the % by weight of creatine in each subunit is similar, then the creatine is said to be substantially uniformly dispersed throughout the product. One skilled in the art will appreciate that the % by weight need not be identical for each subunit to indicate uniform dispersion. In accordance with the present invention, the % by weight of creatine for each subunit of the final delivery system varies by less than 2%. In one embodiment, the % by weight of creatine for each subunit of the final delivery system varies by less than 1.5%. In another embodiment, the % by weight of creatine for each subunit varies by less than 1%. In another embodiment, the % by weight of creatine for each subunit varies by less than 0.5%.

Similarly, the degradation of the creatine and other bioactives can be determined by standard analytical techniques taking into account the total amount of each compound included in the preparation of the matrix. Many bioactives degrade to yield specific breakdown products, the presence or absence of which can be determined in the final product. For example, creatine is hydrolysed to creatinine, which can be distinguished from creatine using chromatographic techniques, such as HPLC. As indicated above the degradation of the creatine and other bioactives is minimised during the preparation of the delivery system and is less than about 20% in the final product.

Water activity (a_{w}) of the final product can also be analysed by standard techniques. The a_{w} of a food product is a physical property that has direct implications on the microbial safety of the product and influences storage stability. Lower a_{w} values generally indicate a food product that is more stable and more resistant to microbial contamination than one with a high a_{w} value due to the requirement for water of most microbes and the fact that most deteriorative processes in food products are mediated by water. As is known in the art, the a_{w} value of a food product is the ratio of the water vapour pressure of the product (p) to that of pure water (pₒ) at the same temperature, i.e. a_{w}=p/pₒ. In accordance with the present invention, the water activity of the final delivery system is less than about 0.7.

In addition, the delivery system may undergo testing to evaluate such factors as the microbial content of the product and the shelf-life of the product. Such quality control testing is standard in the art and can be conducted using known methods.

For example, microbial analysis of the delivery system can be conducted using techniques approved by the appropriate regulatory board, such as those described in “The Compendium of Analytical Methods: HPB Methods for the Microbiological Analysis of Foods” issued by the Health Products and Food Branch of Health Canada. Shelf life is typically evaluated using accelerated shelf life tests in which the stability of the system and the degradation of the creatine and bioactives contained therein is analysed under conditions that are known to accelerate the degradation of food products and can be correlated to the stability of the product under normal storage conditions.

Format of the Delivery System

The present invention contemplates various formats for the delivery system. For example, the delivery system may be in the form of a confectionery, such as a jujube, in which case it may be formulated alone or it may further comprise a coating, such as a chocolate or yoghurt coating. Preparation of jujube or jelly type confectionery products are known in the art and include, for example, the use of moulds, injection-filling of pre-formed packages and extrusion processes. It will be readily apparent to one skilled in the art that such standard techniques can be applied to prepare a wide variety of different shaped confectioneries.

For example, a variety of differently shaped moulds or preformed packages may be used. Jelly candies such as imitation fruit pieces, fruit bars, and sugared jellies are typical. These confections have a firm, but soft, texture that contributes to their desirable mouth feel. Jelly candies are typically manufactured by the Mogul system in which starch moulds are formed by making a plurality of depressions of the desired shape in a bed of starch. In the Mogul system, the ingredients are blended at the appropriate temperature and then the liquid mixture is deposited into the starch mould, which forms the confection and helps to reduce the moisture content. The deposited confections are typically dried for about 24-72 hours to reach the desired moisture content of about 10% to 30% by weight. Jelly candies can also be manufactured by injection-filling packages pre-formed into an appropriate size and shape with the liquid mixture and allowing the mixture to set up.

Alternatively, the delivery system can be formed as confectionery products by an extrusion process in which the matrix mass is forced at relatively low pressure through a die which confers on the matrix the desired shape and then the resultant extrudate is cut off at an appropriate position to yield products of the desired weight. For example, the matrix can be forced through a die of relatively small cross-section to form a ribbon, which is carried on a belt under a guillotine-type cutter which cuts the moving ribbon into pieces of equivalent weight and dimensions. Alternatively, the mass may also be extruded as a sheet, which is then cut with a stamp or cookie type cutter into appropriate shapes. After moulding or shaping, the delivery system confectionery product is moved by a conveyor to an area where it may be further processed or simply packaged.

Methods of making and applying coatings to confectionery products are also well-known in the art. Coatings are in general compound coatings the major ingredients of which are sugar and fat. Flavours and colours are often added. Chocolate coatings are usually based on cocoa butter whereas yoghurt coatings typically comprise powdered yoghurt. In general, the coating material comprises a fat that is solid at room temperature, but liquid at temperatures in excess of, for example, 35° C., together with other materials that confer appropriate organoleptic attributes on the final coating. Typically, application of the coating to the confection takes place while the coating is molten, for example, by passing the formed confection simultaneously through a falling curtain of liquid coating and over a plate or rollers which permit coating to be applied to the under surface of the confection. Excess coating is blown off by means of air jets and the coated confection passes through a cooling tunnel where refrigerated air currents solidify the applied coating. In accordance with the present invention, the properties and method of application of the coating must not interfere with, or compromise, the properties of the delivery system. For example, the application of the coating must not require elevated temperatures that would affect the stability of the bioactive ingredient(s) incorporated into the delivery system.

The present invention further contemplates the delivery system as a filling or a coating, for example, for baked goods such as wafers or cookies. For example, the matrix can be used as a layer between two wafers, or a jelly layer on the top of a cookie or sponge, in which case the product may be further coated with a chocolate or other flavoured coating, if desired, as described above for confectionery products. Alternatively, the matrix may be used to fill doughnut type baked goods. Methods of filling and coating baked goods are also well known in the art.

Method of Administration

The present invention also provides a method of administrating creatine alone or together with other bioactives to an animal in order to enhance muscle size and/or muscle strength and/or to provide health benefits.

The organoleptic properties of the delivery system of the present invention ensure that it is easy to take and/or to administer. In one embodiment, the delivery system is formulated for administration to humans and thus contains flavours that would appeal to humans, such as fruit-based flavours.

In another embodiment, the delivery system is formulated for administration to a non-human animal. In a related embodiment the non-human animal is a domestic animal, such as a dog or a cat. Administration of bioactive ingredients to an animal in conventional solid dosage forms, such as tablets and capsules, can be problematic in that the animal often expels them, and multiple dosing is often difficult because the animal learns to resist the dosing procedure. It will be readily apparent that the delivery system of the present invention, which is formulated as a foodstuff, is ideally suited for administration of bioactive ingredients to animals. When formulated for this purpose, the matrix may contain flavours that more typically appeal to non-human animals, for example, fish or meat flavours. Additional bioactive ingredients more suited to animal use, such as dessicated liver, may also be included.

Due to the efficiency of delivery of the creatine contained therein, the present invention is particularly advantageous for obtaining extra growth in lean muscle mass and strength without undesirable side effects normally associated with consuming dosages of creatine over five grams. Once blood plasma creatine reaches a critical concentration, creatine enters into the muscle fibres as phospho-creatine, which is used by the body as a source of energy and leads to increases in strength and lean muscle mass, .

The effectiveness of the delivery system to provide a prolonged supracritical blood plasma creatine concentration can be demonstrated by comparing increase in plasma creatine concentrations of the matrix versus traditional Creatine supplementation methods. Amounts suitable to allow a subject to maximize its plasma creatine concentration over a prolonged period (for example, approximately 3 hours) are used. The creatine loading phase dosage is estimated from the total creatine storage capacity of the subject, which is related to muscle mass, weight and exercise level. For example, in one traditional method for supplementing the diet of a male athlete, a loading dosage ranging from 12 grams to 25 grams/day is recommended. This dosage can be significantly reduced by using the delivery system of this invention, for example in the form of a jujube, since the plasma creatine levels can be maintained with a lower oral dosage level of creatine provided by the jujube. This oral dosage can also be calculated based on total creatine storage capacity of the subject, muscle mass, weight and exercise level of the subject. Thus, the effective amount of creatine provided by this delivery system that is equivalent to the 12 to 25 grams/day, can be accomplished with one jujube that contains 5-10 grams creatine powder.

For example, a maintenance level of creatine can be provided by the delivery system of the present invention by administration of about 3 to 4 grams creatine/day. To facilitate such administration, the delivery system of the present invention can be formulated to comprise from about 1.5 grams creatine, which can be administered in one or multiple servings per day. Typically, in order to maximise the effect of the creatine in enhancing muscle size and/or strength, the creatine in the delivery system is administered to the athlete without food.

As demonstrated in the literature and from exemplary blood assays, regular creatine monohydrate absorbs at approximately 10-15% when ingested with water. Inone embodiment of the present invention, the delivery system absorbs at about 100%. In another embodiment of the present invention, the delivery system further provides timed release of creatine such that the creatine is released into the blood over a number of hours rather than being quickly absorbed or rapidly broken down.

Kits

The present invention additionally provides for kits containing the delivery system for administration to an animal. The kit would provide an appropriate dosing regimen for a prescribed period for the creatine and other bioactive ingredients optionally contained in the delivery system.

The kits of the invention comprise one or more packages containing the delivery system in combination with a set of instructions, generally written instructions, relating to the use and dosage of the bioactive ingredients contained in the delivery system. The instructions typically include information as to the appropriate dosage and dosing schedule for the bioactive ingredients within the delivery system. The packages containing the delivery system may in the form of unit doses, bulk packages (for example, multi-dose packages) or sub-unit doses. The doses may be packaged in a format such that each dose is associated, for example, with a day of the week. There may also be associated with the kit a notice in the form prescribed by a governmental agency regulating the manufacture, use or sale of biological products, which notice reflects approval by the agency of manufacture, use or sale for human or animal administration.

To gain a better understanding of the invention described herein, the following examples are set forth. It should be understood that these examples are for illustrative purposes only. Therefore, they should not limit the scope of this invention in any way. All percentages throughout the specification and claims are by weight of the final delivery system unless otherwise indicated.

### EXAMPLES

### Example 1

### Exemplary Formulations

Examples of possible formulations of creatine alone or with other bioactive agents suitable for incorporation into the delivery system of the present invention include: (1) creatine alone; (2) creatine with extracts for performance enhancement (e.g. rodiola crenulata mix (from PharmEast)); (3) creatine with extracts for thermogenic enhancement such as a diuretic, metabolic enhancer (extract from PharmEast); (4) creatine with extracts for alertness and mental enhancement such as gingko biloba, phosphatidyl serine or choline, CoQ10; (5) creatine with extracts for muscle enhancement such as solubilized isoflavones; (6) creatine with vitamins and/or minerals and (7) creatine with extracts for general performance enhancement and health such as yohimbe, gingko, puanama muira, and saw palmetto.

### Example 2

### Delivery System for Creatine

One example of a delivery system containing creatine alone is as follows:

| Ingredient | % by Weight |
|---|---|
| Glycerol | 14.82% |
| Propylene Glycol | 5.39% |
| Creatine monohydrate | 11.91% |
| Corn Syrup 62DE | 32.33% |
| Sucralose | 0.04% |
| Modified Starch (Staley Softset ®) | 2.70% |
| Potassium citrate | 2.19% |
| High fructose corn syrup | 9.43% |
| Water | 14.82% |
| Gelatine 100 bloom type B | 1.34% |
| Gelatine 250 bloom type A | 4.04% |
| Gellan (Kelcogel ® LT100) CP Kelco | 0.33% |
| Colour | 0.21% |
| Flavour | 0.46% |
| Total: | 100.00% |

Glycerol and propylene glycol were first blended and the creatine was then added. The blend was heated to 65-70° C. In a separate container, the two types of gelatine and the gellan were blended together. The fructose syrup and water were mixed and heated to 60° C., after which the gelatine:gellan mixture was added. The mixture was then heated to 75° C. to allow the components to dissolve. In a third container, the corn syrup was warmed to 30-35° C. and the sucralose, potassium citrate, and starch were then blended in. The corn syrup mixture was combined with the gelatine:gellan mixture and heated to 75-80° C. until the moisture content was reduced and the desired solids level achieved. The creatine mixture is then added together with the colour and flavour additives. The delivery system is then moulded using standard techniques.

Various additional bioactives may be added up to the matrix. The total amount of creatine and other bioactives will be approximately 25% by weight. Example 1 provides descriptions of possible formulations.

### Example 3

### HPLC Analysis of Creatine Stability

Samples of the delivery system produced by the method described in Example 2 were analyzed by high performance liquid chromatography (HPLC) using UV detection to determine the percentage of creatine. Prior to injection, each sample was subject to a dissolution procedure wherein the sample was cut into small pieces and heated in 400 ml of Type 1 water at 90° C. for 10 minutes. The samples were then transferred to a water bath at 4° C. and 50 ml of 1% perchloric acid was added. The mixture was then heated to 28° C., transferred to a 500 ml volumetric flask and the volume made up to 500 ml with Type 1 water. A 60 μL aliquot of this solution was then added to 140 μL of methanol and vortexed. Three replicates were prepared for each sample. Samples of 10 μL of the final solution were used to inject into the HPLC.

The percentage of creatine (by weight) was determined by comparing the mean response of creatine in each sample to the mean response of a stock solution at known concentrations. For each replicate prepared as described above, the solution was injected in triplicate.

Tables 1 and 2 outline the quantity and percentage creatine in the samples of the delivery system. Of particular note is the only slight variation between the percentage creatine by weight of each jujube despite the larger variation in the weight of the jujubes. The percentage by weight of creatine determined for each jujube varied between 7.71% and 9.04% (% CV=14.1%), while the weight of the jujubes varied from 7082.40 mg to 11124.16 mg. The mean percentage creatine by weight for the samples was 8.0%. This is consistent with the expected amount of 9% of chelate in the final product.

### Example 4

### In vivo Testing of the Delivery System I

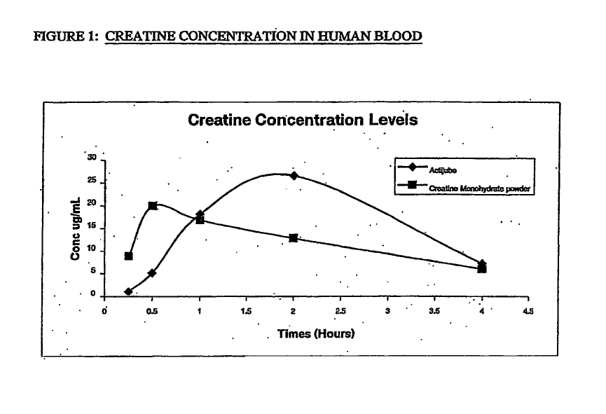

Serum concentration levels of creatine of subjects who ingested either one gram of micronized creatine powder in five ounces of water or one gram of micronized creatine in jujubes (prepared as described in Example 2) were analysed by mass spectroscopy. The samples were taken over a period of four hours. Results are shown in FIG. 1.

In contrast to the results with the creatine powder, creatine ingested by way of the jujube resulted in a greater amount of creatine in the blood system, which would be available to enter the muscle fibres for conversion into energy. Accordingly, the delivery system of the present invention can be beneficial where a continuous flow of creatine into the muscle is desired, for example, during long workout periods. Coupled with the ingestion of the jujubes containing creatine, individuals may also further enhance the absorption of creatine into their muscles by consuming beverages containing additives such as, but not limited to, arginine, which have been shown to increase the uptake of creatine.

### Example 5

### In vivo Testing of the Delivery System II

Human serum concentration levels of creatine in subjects who ingested jujubes prepared as described in Example 2 were analysed by HPLC using mass spectroscopy (MS) detection.

In one study, during a period of four days, serum samples from one subject who consumed either (1) 1 gm of creatine monohydrate in a jujube (Day 1A); (2) 500 mg creatine monohydrate/500 mg creatine chelate in the form of a ‘mixed’ jujube (Day 1B); (3) 1 gm creatine monohydrate powered drink (Day 2A); or (4) 500 mg creatine monohydrate/500 mg creatine chelate powered drink (Day 2B). For the entire study, serum samples were taken over a period of six sampling times. The subject fasted for eight hours prior to dosing.

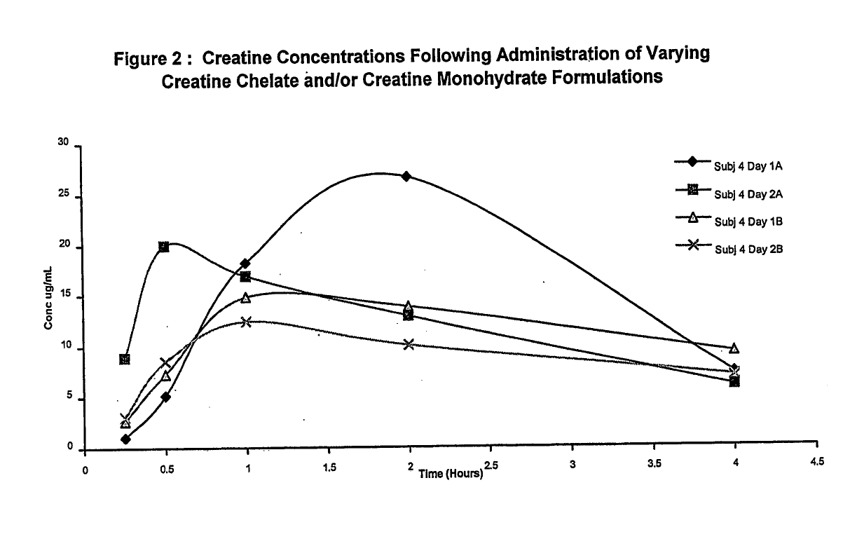

Samples were stored at −20° C.±10° C. for the duration of the analysis. The serum samples were prepared by first adding 50 μL of an internal standard and 20 μL of a 50% perchloric acid solution to 250 μL of the sample, after which they were centrifuged. The supernatant of each sample was then injected into the HPLC/MS system for analysis. The results are plotted in FIG. 2.

The results show that higher serum levels of creatine concentrates were achieved when the subject consumed 1 gm of the creatine monohydrate contained in the jujube compared to values obtained when the subject consumed the creatine powered drinks or the ‘mixed’ jujube containing both creatine monohydrate and creatine chelate. Additionally, serum creatine levels were also capable of being maintained for a longer period of time when the subject consumed the jujube containing creatine monohydrate. The higher serum creatine level over a longer period of time was also noted as creatine levels were still elevated after two hours following ingestion of the creatine monohydrate jujube.

### Example 6

### Delivery System for Creatine and Other Bioactive Ingredients

An example of a delivery system containing creatine together with other bioactive ingredients is as follows:

| Ingredient | % by Weight |
|---|---|
| Glycerol | 13.82% |
| Propylene Glycol | 5.53% |
| Creatine monohydrate(CM) | 4.59% |
| Conjugated Linoleic Acid (CLA) | 4.59% |
| Lecithin | 1.05% |
| Isomalt syrup | 33.17% |
| Sucralose | 0.055% |
| Modified Starch (Staley Softset ®) | 2.76% |
| Potassium citrate | 2.24% |
| N,N, dimethylglycine (dmg) | 0.47% |
| Rhodiola/Seabuckthorn extract | 0.21% |
| solution | |
| Chromium chelate | 0.11% |
| High Fructose Corn syrup | 9.68% |
| Water | 15.20% |
| Gelatine 250 bloom type A | 5.53% |
| Gellan (Kelcogel ® LT100) CP | 0.33% |
| Kelco | |
| Colour | 0.08% |
| Flavour | 0.08% |
| Total: | 100.00% |

The CLA, creatine and lecithin were first mixed together. The glycerol and propylene glycol were mixed and heated to 65-70° C. The CLA/creatine/lecithin blend was then added to the solvents and the resultant mixture was maintained at 65-70° C. In another container, the gelatine was mixed with the gellan. The fructose syrup and water were combined and heated to 60° C. and the gelatine:gellan mixture was then added, after which the temperature was raised to 75° C. and maintained at this temperature until the solids dissolved. In another container, the isomalt syrup was warmed to 30-35° C. and the sucralose, citrate, dmg, rhodiola/seabuckthorn extract, chromium chelate and starch were then blended in. This mixture was combined with the gelatine mixture and the temperature maintained at 75-80° C. until the moisture content was reduced sufficiently to give the desired solids level. Once the proper moisture level was achieved, the glycerol-glycol mixture was blended in together with colour and flavouring additives. The mixture was then moulded using standard techniques.

### Example 7

### Delivery System for Creatine

Another example of a delivery system containing creatine is as follows:

| Ingredient | % by Weight |
|---|---|
| Glycerol | 15.97% |
| Propylene Glycol | 5.51% |
| Creatine Monohydrate | 16.71% |
| 63 DE Corn syrup | 21.20% |
| High Fructose Corn Syrup | 24.78% |
| Gelatine 250 Bloom Type A | 5.51% |
| Gellan | 0.33% |
| Sucralose | 0.06% |
| potassium citrate | 1.40% |
| Modified Starch (Staley | 2.75% |
| Miraquick ®) | |
| Water | 4.96% |
| Flavour | 0.56% |
| Colour | 0.28% |
| Total: | 100.00% |

Creatine was added to a mixture of glycerol and propylene glycol, and heated to 40-60° C. The syrups were blended with water and the dry ingredients were mixed into the syrup mixture. The combined mixture was then heated to at least 80° C. Alternatively, the blended dry ingredients can be blended in with simultaneous live steam injection to reach at least 80° C. The solid content was then adjusted by addition water if necessary to provide a final moisture content of between about 10% to about 30%. At this point, the temperature of the syrup mixture was lowered to between 50° C. and 80° C. and the glycerol-glycol mixture was added. Colour and/or flavouring additives were then added and the delivery system was injection filled into the preformed packaging.

Various additional bioactives may be added up to the matrix. The total amount of creatine and other bioactives will be approximately 25% by weight. Example 1 provides descriptions of possible formulations.

### Example 8

### HPLC Analysis of Creatine Stability

Samples of the delivery system produced by the method described in Example 7 were analyzed by HPLC using UV detection to determine the percentage of creatine monohydrate by weight of each sample. Prior to injection, each sample was subject to a dissolution procedure wherein the sample was cut into small pieces and heated in 200 ml of water at 90° C. for 10 minutes, then transferred to a water bath at 4° C. The mixture was subsequently heated to 28° C., transferred to a 250 ml volumetric flask and the volume made up to 250 ml with water. After mixing, a 1 ml aliquot of the mixture was placed into an Eppendorf tube and centrifuged at 10 000 rpm. The supernatant was filtered through a 0.2 μfilter and centrifuged again at 10 000 rpm. A 5 μl sample of the supernatant was then taken for HPLC analysis. Three injections were made for each sample preparation.

The results of the HPLC analysis are given in Tables 3 and 4. Both the weight of the jujubes and the percentage by weight of creatine contained within each sample are notably uniform. The weight of the jujubes varied from 26 262.37 mg to 26 954.56 mg, with an average value of 26 774.37 mg, and the percentage by weight of creatine varied from 11.75% to 11.85%, with an average value of 11.80%.

### Example 9

### Accelerated Shelf-Life Determination

An accelerated shelf life test was conducted on the creatine delivery system prepared by the method described in Example 7. Microbial analysis was conducted using approved methods as described in The Compendium of Analytical Methods: HPB Methods for the Microbiological Analysis of Foods (Volume 2) issued by the Health Products and Food Branch of Health Canada. After subjecting samples of the delivery system to a temperature of 35° C. and a relative humidity of 45-55% for a period of 35 days, the samples were tested for the presence of various microorganisms as listed in Table 5. The average water activity of the samples tested was approximately 0.51.

In addition to the above microbial analysis, the creatine level in each sample was determined by HPLC prior to the test and after 35 days. The average creatine content for four samples randomly selected for analysis after 35 days was compared to the average creatine content for three samples taken prior to the shelf life test. HPLC analysis of creatine monohydrate levels was conducted as described in Example 8.

The results, as shown in Table 5, indicate that after a period of 35 days at the above-described conditions, microbial contamination was minimal and well below accepted levels. Based on these results, the delivery system is shown to have a stable shelf life of at least one year from the date of manufacture.

Results from the HPLC analysis also indicated that levels of creatine monohydrate remained stable in the jujubes after 35 days exposure to the above-described conditions. Prior to the start of the experiment, three jujubes had an average of 13.4% by weight of creatine monohydrate. After 35 days, four jujubes were shown to have an average of 14.2% by weight of creatine monohydrate, which is within the error limits of the analysis performed.

### Example 10

### Analysis of Water Activity of the Delivery System

Water activity was measured in samples of jujubes that had been prepared according to the method described in Example 7.

The procedure for measuring water activity is based on the fact that the water activity of a sample is equal to the relative humidity created by the sample in a closed environment when in equilibrium. The procedure uses a water activity meter constructed by David Brookman & Associates (DB&A). The DB&A Water Activity Meter uses an Omega Engineering HX92C Relative Humidity indicator to measure the relative humidity within a closed environment containing the sample. The Omega probe converts the relative humidity (R.H.) into milliamperes (ma), where 4 ma equals 0% R.H. and 20 ma equals 100% R.H. The water activity meter is calibrated to 11.3% R.H. using a saturated solution of LiCl and to 75.3% R.H. using a saturated solution of NaCl.

The samples are manually macerated in a plastic bag and then transferred to a 30 ml sample bottle. The bottles are filled with sample to at least 1 cm from the shoulder. The bottles are capped until use and stored at room temperature. Measurements are taken by screwing the sample bottle onto the DB&A meter probe and the bottle probe assembly is maintained in a vertical position in a rack. Measurements are taken at hourly intervals at room temperature (20-22° C.) until such time that successive readings do not vary more than 1%.

Random sampling of the jujubes was conducted. The water activity (a_{w}) was determined to be 0.507, 0.515 and 0.544. These values are well below levels those that favour the growth of microorganisms. It has been shown that microorganisms generally grow best between a_{w} values of 0.995-0.980 and most microbes will cease to grow at a_{w} values less than 0.900.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

TABLE 1
PEAK HEIGHT RESPONSES AND DETERMINED QUANTITY (mg) OF CREATINE CHELATE IN
JUJUBES

| | Reference Stock | Jujube No. | | | | | | | | |
|---|---|---|---|---|---|---|---|---|---|---|
| | Peak Height | (Peak Height Response) | | | | | | | | |
| | Response | 1 | 2 | 3 | 4 | 5 | 6 | 7 | 8 | 9 |
| | 394.09 | 452.48 | 570.96 | 589.83 | 622.90 | 600.57 | 477.41 | 618.16 | 530.70 | 648.05 |
| | 388.77 | 481.39 | 563.36 | 602.88 | 635.36 | 633.99 | 488.51 | 628.59 | 537.26 | 649.14 |
| | 385.00 | 505.71 | 601.46 | 598.41 | 636.37 | 648.53 | 457.92 | 615.64 | 527.72 | 630.77 |
| MEAN | 389.29 | 479.86 | 578.59 | 597.04 | 631.54 | 627.03 | 474.61 | 620.80 | 531.89 | 642.65 |
| S.D. | 4.57 | 26.65 | 20.16 | 6.63 | 7.50 | 24.36 | 15.49 | 6.87 | 4.88 | 10.31 |
| % CV | 1.2 | 5.6 | 3.5 | 1.1 | 1.2 | 3.9 | 3.3 | 1.1 | 0.9 | 1.6 |

| | | | | | | | | | |
|---|---|---|---|---|---|---|---|---|---|
| Determined Quantity of | 640.37 | 772.13 | 796.75 | 842.79 | 836.77 | 633.37 | 828.45 | 709.81 | 857.62 |
| Creatine Chelate in Jujube | | | | | | | | | |
| (mg)¹ | | | | | | | | | |

¹Calculated as the (Mean Peak Height of Jujube Solutions)/(Mean Peak Height of Reference Stock Solutions) × (1039 μg/mL) × (500 mL)/(1000)

TABLE 2
PERCENTAGE CREATINE CHELATE
BY WEIGHT IN JUJUBES

| | | Determined | |
|---|---|---|---|
| | | Concentration of | |
| | | Creatine Chelate | % Creatine Chelate |
| Jujube No. | Weight (mg) | (mg) | by Weight (%) |
| 1 | 7082.40 | 640.37 | 9.04 |
| 2 | 9620.96 | 772.13 | 8.03 |
| 3 | 10299.80 | 796.75 | 7.74 |
| 4 | 10583.38 | 842.79 | 7.96 |
| 5 | 10535.61 | 836.77 | 7.94 |
| 6 | 7895.14 | 633.37 | 8.02 |
| 7 | 10434.55 | 828.45 | 7.94 |
| 8 | 9095.45 | 709.81 | 7.80 |
| 9 | 11124.16 | 857.62 | 7.71 |
| MEAN | 9630.16 | 768.67 | 8.02 |
| S.D. | 1362.14 | 87.07 | 0.40 |
| % CV | 14.1 | 11.3 | 5.0 |

TABLE 3
Percentage Creatine Monohydrate by weight in Jujubes

| | | Determined | |
|---|---|---|---|
| | | Conc. of | |
| Jujubes | Weight/mg | Creatine/mg | % Creatine by weight |
| 1 | 26954.56 | 3175.55 | 11.78% |
| 2 | 26262.37 | 3110.82 | 11.85% |
| 3 | 25807.23 | 3151.85 | 11.75% |
| 4 | 28925.42 | 3181.04 | 11.81% |
| 5 | 26848.04 | 3168.55 | 11.80% |
| 6 | 26847.58 | 3165.65 | 11.80% |
| Average | 26774.37 | 3159.41 | 11.80% |

TABLE 4
Peak Height Responses of Creatine Monohydrate in Jujubes

| | Peak Area | | | | | |
|---|---|---|---|---|---|---|
| | No. 22 | No. 23 | No. 24 | No. 25 | No. 15 | No. 27 |
| | Jujube 1 | Jujube 2 | Jujube 3 | Jujube 4 | Jujube 5 | Jujube 6 |
| | 25051.20 | 24550.57 | 24829.29 | 25080.93 | 25031.10 | 25010.23 |
| | 25977.39 | 24559.88 | 24921.40 | 25137.22 | 25023.13 | 25027.83 |
| | 25105.90 | 24591.11 | 24922.88 | 25147.54 | 25014.97 | 25024.65 |
| Average | 25078.50 | 24567.18 | 24897.19 | 25121.76 | 25023.07 | 25023.94 |
| Std. Dev | 27.87 | 21.24 | 53.02 | 35.71 | 8.07 | 4.39 |
| CV | 0.1% | 0.1% | 0.2% | 0.1% | 0.0% | 0.0% |

TABLE 5
Microbial Analysis of Creatine Monohydrate
Jujubes - Accelerated Shelf Life Determination
Water activity: approximately 0.51
Time: 35 days
Temperature: 35° C.
Humidity: 45-55%

| | HPB REFERENCE | RESULTS (No. |
|---|---|---|
| TEST CONDUCTED | NUMBER | Colonies/gm product) |
| Total aerobic plate count | MFHPB-18 | <10 |
| Total coliforms | MFHPB-34 | <10 |
| E. Coli | MFHPB-34 | <10 |
| Yeast | MFHPB-22 | <50 |
| Mould | MFHPB-22 | <50 |
| Yeast Osmophilic | MFHPB-22 | <50 |
| Mould Osmophilic | MFHPB-22 | <50 |
| Staphylococcus aureus | MFHPB-21 | <25 |
| Salmonella | MFHPB-20 | not detected |

The embodiments of the invention in which an exclusive property or privilege is claimed are defined as follows:

1. An oral delivery system for creatine formulations comprising:
a) one or more sources of creatine, and
b) a matrix comprising:
i) a sugar component comprising one or more sugars, sugar syrups, sugar alcohols or a combination thereof;
ii) one or more low set temperature carbohydrates or a modified version thereof;
iii) a hydrocolloid component comprising one or more hydrocolloids;
iv) a solvent component comprising one or more polyhydric alcohols;
v) one or more sources of mono- or divalent cations, and
vi) one or more sources of water,
wherein said creatine is substantially uniformly dispersed in said matrix and wherein said delivery system is a semi-solid at room temperature and has a final moisture content of between about 15% and about 30% by weight and a water activity of less than about 0.7.

2. The delivery system according to claim 1, wherein said matrix has a final pH between about 6.0 and 7.5.

3. The delivery system according to claim 1 or 2, wherein said matrix remains flowable at or above a temperature of 45° C.

4. The delivery system according to any one of claims 1-3, wherein said one or more carbohydrate comprises a starch or a modified version thereof.

5. The delivery system according to any one of claims 1-4, wherein said hydrocolloid component comprises gelatine, gellan or a mixture thereof.

6. The delivery system according to any one of claims 1-5, wherein said sugar component comprises one or more sugar syrup.

7. The delivery system according to any one of claims 1-6, wherein said solvent component comprises glycerol, a short chain polyalkylene glycol or a combination thereof.

8. The delivery system according to any one of claims 1-7, further comprising a sweetener, a buffer, a natural or artificial flavouring, a colouring agent or a combination thereof.

9. The delivery system according to any one of claims 1-8, further comprising one or more bioactive ingredients.

10. The delivery system according to claim 9, wherein said one or more bioactive ingredients are selected from the group of drugs, botanicals, nutritional supplements, vitamins, minerals, enzymes, hormones, proteins, polypeptides and antigens.

11. A method of making a delivery system for creatine formulations comprising:
(a) preparing a blend of one or more carbohydrate, one or more hydrocolloid, one or more sugar, sugar alcohol or sugar syrup, or a combination thereof, one or more sources of mono- or divalent cations, and water;
(b) heating said blend to a temperature of less than 100° C.;
(c) maintaining said blend at a temperature of less than 100° C.;
(d) adjusting the moisture content of the blend to between about 15% and about 30% by weight;
(e) preparing a solution of one or more creatine compound or analogue thereof in a solvent component comprising one or more polyhydric alcohols at or below a temperature of 70° C.;
(f) combining said blend and said solution of creatine at a temperature of 70° C. or below to form a matrix whereby the creatine is substantially uniformly dispersed throughout said matrix, and
(g) forming said matrix into shapes,
wherein the delivery system has a final moisture content between about 15% and about 30% and a water activity of less that about 0.7.

12. The method according to claim 11, wherein said blend is maintained at a temperature of between about 75° C. and about 80° C. in step (c).

13. The method according to claim 11, wherein said blend is maintained at a temperature of between about 80° C, and about 100° C. in step (c).

14. The method according to any one of claims 11-13, wherein said matrix has a final pH between about 6.0 and 7.5.

15. The method according to any one of claims 11-14, wherein said one or more carbohydrate comprises a starch or a modified version thereof.

16. The method according to any one of claims 11-15, wherein said hydrocolloid component comprises gelatine, gellan or a mixture thereof.

17. The method according to any one of claims 11-16, wherein said sugar component comprises one or more sugar syrup.

18. The method according to any one of claims 11-17, wherein said solvent component comprises glycerol, a short chain polyalkylene glycol or a combination thereof.

19. The method according to any one of claims 11-18, wherein a natural or artificial flavouring, a colouring agent or a combination thereof is added to the matrix in step (f).

20. The method according to any one of claims 11-19, wherein said blend further comprises a sweetener, a buffer or a combination thereof.

21. The method according to any one of claims 11-20, wherein one or more bioactive ingredients is added to the matrix in step (f).

22. The method according to claim 21, wherein said one or more bioactive ingredients are selected from the group of drugs, botanicals, nutritional supplements, vitamins, minerals, enzymes, hormones, proteins, polypeptides and antigens.

23. A delivery system for creatine prepared by the process according to any one of claim 12-22.

24. Use of a delivery system according to any one of claims 1-9 for oral administration of creatine to an animal in need thereof.
